(19) United States
(12) Patent Application Publication
(10) Pub. No.: US 2015/0201888 A1
(43) Pub. Date: Jul. 23, 2015

(54) WELLNESS INCENTIVE METHOD AND DEVICE

(71) Applicants: Gregory C. Vachon, Oak Park, IL (US); Charlie Zei, Chicago, IL (US)

(72) Inventors: Gregory C. Vachon, Oak Park, IL (US); Charlie Zei, Chicago, IL (US)

(21) Appl. No.: 14/138,529

(22) Filed: Dec. 23, 2013

Related U.S. Application Data
(60) Provisional application No. 61/746,004, filed on Dec. 26, 2012.

(51) Int. Cl. A61B 5/00; A61B 5/02; A61B 5/145; A61B 5/021

(52) U.S. Cl. CPC A61B 5/7278; A61B 5/021; A61B 5/02028; A61B 5/14546; A61B 5/4845; A61B 5/4884

(57) ABSTRACT

An improved method for incentivizing wellness in employees and a device for implementing the method are provided. The wellness incentive method measures baseline wellness factors of a user while the user performs biometric testing. The baseline wellness factors are then used to calculate a wellness score and baseline incentive amount, both of which are proportional to the risk for future adverse health events. The user retakes the biometric test at a later date and current wellness factors are then measured. The current wellness factors are used to calculate a current wellness score. If the current wellness score improves, an additional incentive is provided to the user which is directly linked to the reduced risk for future adverse health events that the improvement conveys and the difficulty of the change. Other advantageous features include short interval to a cash equivalent payment, significant bonus amounts for incremental improvements, bonus reduction patterns that provoke loss principles of behavioral economics and individualized messaging and payments. These features contribute to the unique effectiveness of the method. A device for measuring a user's wellness factors, particularly fitness, is also provided. A user performs a specific pattern of exercise on the device and data corresponding to wellness factors are obtained. The device may also communicate the data corresponding to wellness factors to a server which calculates a wellness score and presents information to the user.

### CROSS-REFERENCE TO RELATED APPLICATION

This application claims priority to U.S. Provisional Application Ser. No. 61/746,004 filed Dec. 26, 2012

### BACKGROUND OF THE INVENTION

Employee wellness is of importance to employers and employees alike. Without adequate employee wellness, an employer may incur increased costs associated with employee healthcare costs, lost employee productivity, employee absence, higher risk of accidents, and other factors. For employees, being un-well may result in higher out-of-pocket healthcare and prescription costs, and Ultimately, increased likelihood of disease and death.

It is important to both employers and employees for employees to maintain or improve their health and wellness. There are numerous ways for employers to promote wellness in their employees. One way to help employees achieve increased health and wellness is to promote behavior modification. However, behavior modification is not always simple. For behavior modification to work, the employee most see a tangible benefit for making the behavior modification, such as a monetary incentive, that outweighs any interest in maintaining their current lifestyle.

Providing incentives for behavior modification can be effective, but still may have drawbacks if not executed effectively. First, the incentive may only result in a temporary improvement in employee wellness if the incentive is only awarded once, as an employee may revert back to their previous lifestyle after achieving a goal or completing an activity and receiving the incentive. Second, if the reward is not linked to relevant measures, the incentive will fail to drive behavior modification in meaningful areas. Third, if the incentive system is not based on objectively-verified measurements, it may be much more susceptible to gaming. Fourth, if the incentive system does not incorporate known principles of behavioral economic theory, it may prove less effective for driving behavior change. Finally, if the incentive system is not tailored to the individual based on his or her baseline wellness, it may fail to resonate with employees. Accordingly, a recurring tangible benefit model that is tailored to individuals, effectively incorporates behavioral economic theory, and objectively assesses and rewards meaningful incremental improvements in relevant wellness measures may be desired.

An employee wellness incentive system is both more effective and fair when the incentives given to employees for verified improvements in wellness are based on progress. Rather than incentives for meeting a particular threshold, small incremental changes are rewarded with a meaningfull, tangible incentive. By using a progress-based incentive system, employees may be spurred on both by the intrinsic reward of improvements in health (feeling better) and in tangible rewards (cash or cash equivalents) for improvement. This solution avoids the ineffectiveness of behavior modification programs with only a one-time incentive because an employee in a progress-based behavior modification program has an interest in continued improvement in health and wellness.

Unlike a progress-based incentive system, some behavior modification programs reward mere participation in the program and completion of required activities. The drawbacks to this style of program are clear. By merely rewarding participation and activities, the program may be gamed and incentives may be paid out despite a lack of measureable improvement in the wellness of an employee. Further, if the incentive for participation or engagement is a one-time incentive, an employee may not continue any work toward improving wellness after the incentive is provided.

Employee wellness incentive systems are also susceptible to gaining via use of self-reported data. Participants in programs that reward incentives based on self-reported measurements or activities may be tempted to falsify these measurements or activities in order to attain the available incentive.

Some employers, insurance companies and employee benefit administrators implement high employee insurance premium contributions and offer credits back for healthy lifestyle factors. However, these implemented systems are not well designed to incent changes for several reasons. Firstly, they focus on an all-or-none approach to incentives (thresholds, as opposed to progress). Secondly, the discounts on premiums provided occur in the year following the assessment (not timely). Thirdly, they provide very infrequent reinforcement (change in the incentive amount with corresponding messaging). In these programs, employees are rewarded for meeting specific criteria (e.g., BMI<28 and non-smoker) during a one-time annual assessment. Employees who fail to meet these criteria do not receive the reward and must wait until the following year to qualify.

An employee wellness incentive system is more effective when there is a direct link between the incentive amount and the health outcomes of meaningful wellness improvements, such as reduction in risk for future adverse health events. This link enables participants to more easily see the value of incremental wellness improvements and how improvements in some areas have a greater impact on overall health than improvements in other areas.

An employee wellness incentive system is more effective when principles of behavioral economics are incorporated into the model. These principles include loss aversion and meaningfully large bonuses or penalties to drive ongoing improvement. Programs that do not incorporate such principles may fail to realize the maximal effect of the incentive.

An employee wellness incentive program is more effective when it is tailored to the individual participant based on his or her baseline wellness. This can be achieved in several ways: targeted messaging to identify the improvement areas that would most benefit the individual in terms of health and incentive reward; adjusting goals based on the individual's baseline results; and rewarding bonuses that reflect how difficult it is to improve specific wellness factors through behavior change.

Both employers and employees have a vested interest in improving employee wellness. There exists a need for an individually-tailored, progress-based wellness incentive program that provides tangible, recurring incentives to employees for verified improvements in relevant wellness factors and effectively incorporates principles of behavioral economics to drive behavior change.

### SUMMARY OF THE INVENTION

The present disclosure provides a novel wellness incentive method and device for use in the wellness incentive method.

In an embodiment, a wellness incentive method is provided that includes performing a baseline wellness assessment on a user (focused on factors that are meaningful in terms of independent impact on future health outcomes, modifiable by the user and are objectively measurable), analyzing liveliness factors based on the baseline assessment, generating a baseline wellness score from the analyzed wellness factors (linked to risk for future adverse health outcomes), providing a monetary incentive (directly linked to the score) and individually tailored messaging to the user based on their baseline wellness score, performing a subsequent wellness assessment at a later date, analyzing wellness factors based on the subsequent assessment, generating a subsequent wellness score from the analyzed subsequent assessment, comparing the baseline wellness score to the subsequent wellness score; and providing an additional monetary incentive (or penalty) to the user that is directly tied to improvements in individual factors and the characteristics of those factors in that individual (the impact on future health events of an individual's change in score and degree of difficulty generally encountered when making that change in the particular wellness factor at the particular value from which the individual started).

In another embodiment, a wellness testing system is provided, including a stepper device and a processor connected to the stepper device, configured to measure fitness data of a user, analyze wellness factors based on the measured fitness related data, and generate a user fitness and wellness score using the measured and analyzed fitness related data.

In another embodiment, a computer is provided for supporting an internet-based portal which performs operations of receiving wellness data associated with a user, presenting the user with information about the user's wellness, tracking incentives related to user goals, displaying the user goals and progress towards the user goals, displaying a personalized evaluation of the user's goals and encouragement for the user to continue towards the user's goal, and presenting the user with additional user goals that may be selected by the user.

The wellness inventive method and device for implementing the wellness incentive method of the present disclosure has numerous advantages. First, it is an advantage of the disclosure that the wellness incentive method promotes the progress of employees towards better health and provides a system for continuously encouraging the employees to maintain a healthy lifestyle.

Second, the wellness incentive method of the present disclosure has the advantage of providing tangible, recurring incentives to employees for verified improvements in wellness.

Another advantage of the present disclosure is that an employer may see reduced health insurance and lost productivity expenses related to these verified improvements in wellness factors.

Next, it is advantageous that the wellness incentive method of the present disclosure is based on simple, direct, recurring algorithms relating wellness factors to risk for future adverse health events and is therefore less complex for employers and employees to interpret and more easily implemented than previous systems.

Further, the performance testing device may be easily installed and used so that the performance testing device may be conveniently located for users.

Additionally, the performance test of the present disclosure is not complex and may be administered by anyone trained to administer the test.

Additional features and advantages are described herein and will be apparent, the following Detailed Description and figures.

### BRIEF DESCRIPTION OF THE DRAWINGS

FIG. 1 is a flowchart showing an embodiment of the process of the wellness incentive method of the present disclosure.

FIG. 2 is a front elevation view of an embodiment of a stepper device used to practice the method of the present disclosure.

FIG. 3 is a system view of an embodiment of a wellness incentive service of the present disclosure.

FIG. 4 is a view of an embodiment of a welcome page of the client portal of the present disclosure.

FIG. 5 is a view of an embodiment of a goals page of the client portal of the present disclosure.

FIG. 6 is a front elevation view of an embodiment of a fitness testing device used to practice the method of the present disclosure.

### DETAILED DESCRIPTION OF THE INVENTION

In accordance with the present disclosure, a novel wellness incentive method and device have been developed. While the disclosure is described in the context of several embodiments, it will be understood that the disclosure is not limited to any particular embodiment.

The present disclosure provides a business-friendly system for incentivizing the health and wellness of employees. By implementing the present disclosure, an employer may see reduced employee health insurance costs and reduced costs associated with lost time from employee's absence. Employees may see improved health and wellness, and may receive tangible, recurring incentives for the improvement of their health and liveliness. Accordingly, the present disclosure recognizes that both employers and employees have a vested interest in the health and wellness of employees, and provides benefits to both employers and employees through its use.

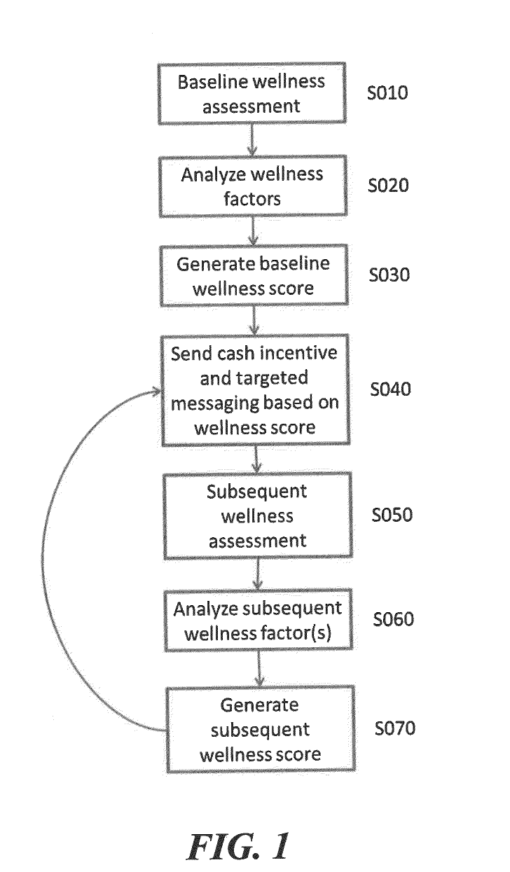

In an embodiment shown in FIG. 1, the method includes conducting a baseline wellness assessment that analyzes wellness factors to generate a baseline liveliness score. Incentives are given out based on the user's baseline score. Goals for improvement may be set based on the baseline wellness score. Subsequent performance tests are then conducted and the same wellness factors are analyzed to generate a subsequent wellness score. The baseline wellness score and the subsequent wellness score are compared to see if scores have improved. If scores are improved, then additional incentives are given out. The method is then repeated at the subsequent testing step.

More specifically, with respect to FIG. 1, a baseline wellness assessment is performed at step S010. The baseline wellness assessment is a physical performance test during which wellness factors which meet the criteria (objectively assessable, modifiable by employee, and meaningful in independent impact on future health events) are measured. In one embodiment the wellness factors may be the participant's hod mass index (BMI, which is mass divided by height squared), blood pressure, cardiovascular fitness, cholesterol, and nicotine use. Each wellness factor is measured individually. After the wellness data measurements are taken, the wellness factors are analyzed at step S020. At step S030, a baseline wellness score is generated using the measured wellness data. A baseline incentive is awarded based on the baseline wellness score at step S040. This incentive is accompanied by individually-tailored messaging that is delivered by email, text messaging, post mail, or other means. This individually-tailored messaging (constructed with algorithms) informs participants how much incentive they are receiving, how they can earn more in subsequent payouts, and what they can do to improve their health.

At step S050, a subsequent wellness assessment is performed. In one embodiment, subsequent wellness assessments may be performed at intervals that are at the discretion of the employee, though with minimal intervals as medically indicated (and, in terms of cost, as negotiated with the employer). After the subsequent wellness assessment is performed, the wellness data obtained is analyzed as step S060. The analyzed wellness data is then used to calculate a subsequent wellness score, as shown at step S070.

After generating the subsequent wellness score, an incentive payment is provided in step S040 that reflects the new wellness score and includes bonuses or penalties for changes in wellness factors. The bonus (or penalty) amount is directly linked to the wellness factor's impact on the risk of future health events (in the particular individual) and the degree of difficulty in making changes in the wellness factor (on average in individuals that have that particular individual's wellness factor profile). This incentive is accompanied by individually-tailored messaging that is delivered by email, text messaging, post mail, or other means. This messaging informs participants how much incentive they are receiving, how they can earn more in subsequent payouts, and what they can do to improve their health.

The incentive given out is monetary (cash or cash equivalent) and is of sufficient magnitude to be greater than two percent of the individual's annual income for a majority of employees in one embodiment. A website is provided for each individual to create and track their wellness scores, incentive payments and goals. The performance test is intentionally simple to administer so that a medical doctor is not needed and the test can be administered by anyone trained to do so. For example, a pharmacist, technician, or medical assistant could perform all the needed factor tests and submit the wellness factor measurements to a software program or website.

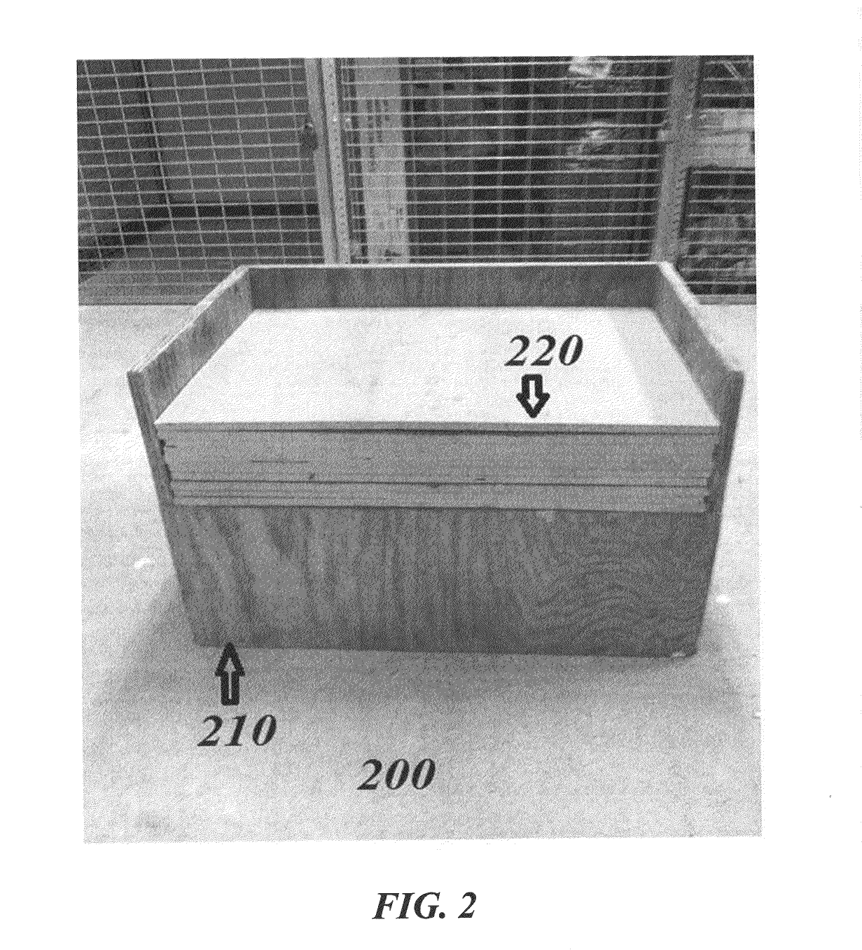

In an embodiment as shown in FIG. 2, the testing device may include a stepper platform 200 onto which a user steps up and down, resulting in a performance test of cardiovascular fitness. The stepper platform 200 includes a base 210 in the form of a horizontal platform with removable height increments 220 stacked inside covering the dimensions of the base so that the height of the step can be easily adjusted. The removable increments 220 may be added or removed based on the height of the user performing the test. The stepper platform 200 may be located at convenient locations such a retail stores or on-site at a business location.

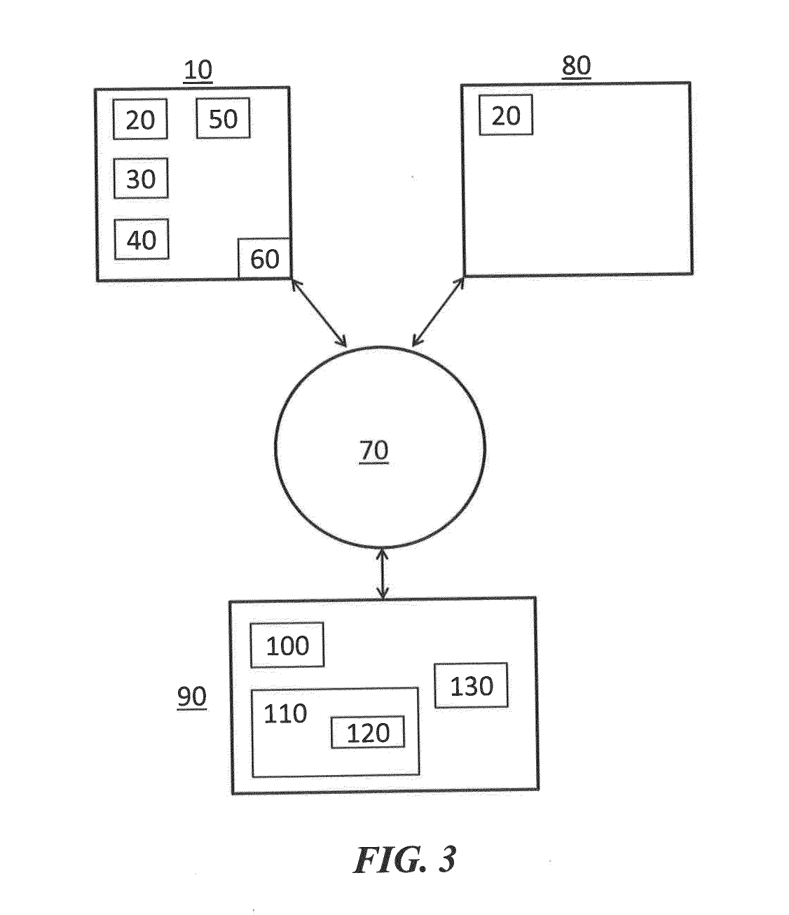

In an embodiment as shown in FIG. 3, the cardiovascular fitness testing device 10 may include stepper 200, client software 20, CPU 30, memory 40, storage 50, and network interface 60. The stepper platform 200 may include the wellness testing device 10 or may be connected to the liveliness testing device 10. Client software 20 may be preinstalled on wellness testing device 10, or may be accessed via network interface 60 and network 70 during testing. For example, client software 20 may be a webpage that is downloaded by wellness testing device 10 from server 90. The wellness testing device 10 may use the CPU 30 and memory 40 to execute the client software 20. The client software 20 may optionally be stored at storage 50 of the wellness testing device 10.

As a user performs a fitness test, wellness data may be automatically obtained and recorded by cardiovascular fitness testing device 10 or may be obtained and recorded by a test administrator. The recorded wellness data may then be submitted by the client software 20 to the server 90 via the network interface 60 and network 70. The network 70 may be any network that interconnects one or more devices. For example, network 70 may be a wired network or a wireless network. Additionally, the network 70 may be the internet, may be connected to the internet, or may be a private intranet.

The wellness data associated with a user's wellness test in one embodiment may be simultaneously obtained and stored in storage 50 of the wellness testing device 10. Upon completing the wellness test, the user may select to submit the wellness data to the employee wellness database 100 located at server 90. Alternatively, the submission of user wellness data to employee wellness database 100 may be performed automatically without user intervention.

The user may perform the wellness test in the presence of an administrator in an alternate embodiment. The administrator may verify the user's identity, verify that the wellness test is being properly performed by the user, and/or obtain user wellness data during the wellness test. Upon completion of the wellness test, the administrator may submit user wellness data to the employee wellness database 100 using client software 20 of the wellness testing device 10.

Alternatively, the administrator may submit the user wellness data using computer $0. Computer 80 may also utilize client software 20 to submit user wellness data to employee wellness database 100 via network 70. Computer 80 may be any type of computing device capable of executing client software 20. For example, computer 80 may be a desktop computer, laptop computer, smartphone, cellular phone, PDA (Personal Digital Assistant), handheld computing device, etc. that may access server 90 via network 70.

In a preferred embodiment, server 90 includes employee wellness database 100 and wellness score engine 110. Wellness score engine 110 includes wellness algorithm 120. Upon receiving user wellness data, server 90 stores the user wellness data in the employee wellness database 100 and calculates a wellness score, as well as individualized incentives and messaging, for the user wellness data using algorithm 120. The generated wellness score is then stored in the member wellness score database 130.

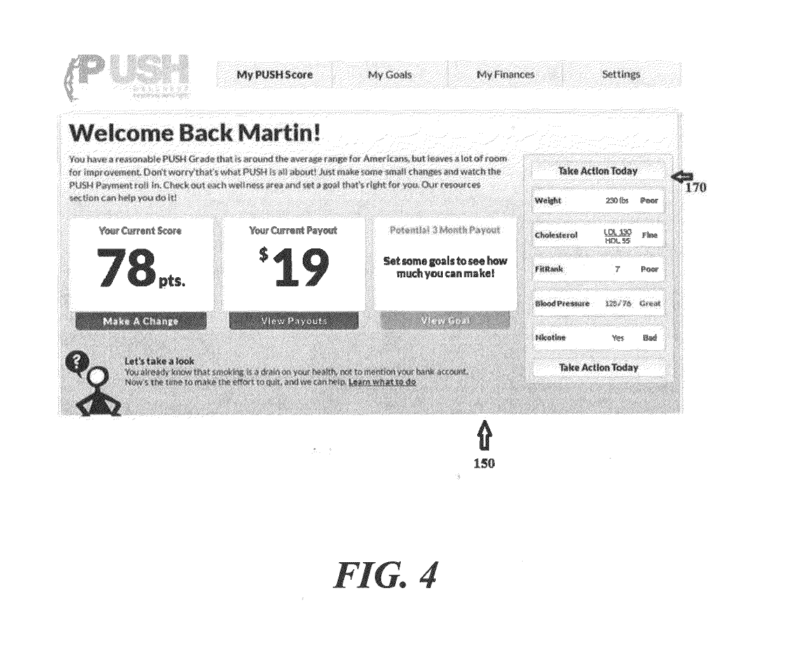

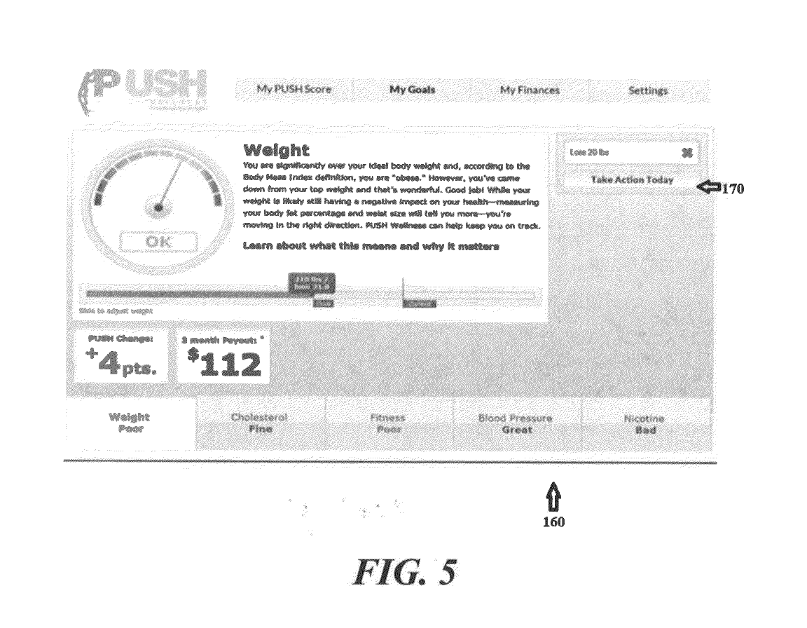

As shown in FIGS. 4 and 5, a user may access a client portal 140 at server 90. The client portal 140 is preferably linked to an individual account for each user and presents a personalized interface. FIG. 4 shows a welcome screen 150 displaying wellness data. The welcome screen 150 may display at least a portion of a user's wellness data and calculated wellness score. Additionally, the welcome screen 150 may display a user's incentive payout information. Information conveyed on the welcome screen 150 may presented as text, charts, graphics, or the like. Using the welcome screen 150, a user may, for example, access their wellness score, goals, financial information, and account settings. The welcome screen 150 may also present optional goals for a user to select for attempting to obtain additional incentives.

FIG. 5 shows a goal screen 160. The goal screen 160 may display a visual representation of a user's goals as well as any progress towards the user's goals. Information conveyed on the goal screen 160 may presented as text, charts, graphics, or the like. The goal screen 160 may display a personalized evaluation of the user's goal and current progress towards the goal, as well as encouragement to continue their progress. The goal screen 160 may additionally present, for example, any change in the user's calculated wellness score, any incentive payout information, and an easy to understand breakdown of the user's wellness data.

As shown in FIGS. 4 and 5, the client portal may include a take action button 170. The take action button 170 may allow the user to quickly select a new goal for additional incentives and this goal-setting may be used in the algorithmic production of individualized messaging. As shown in FIG. 5, a goal may be automatically suggested by the server 90.

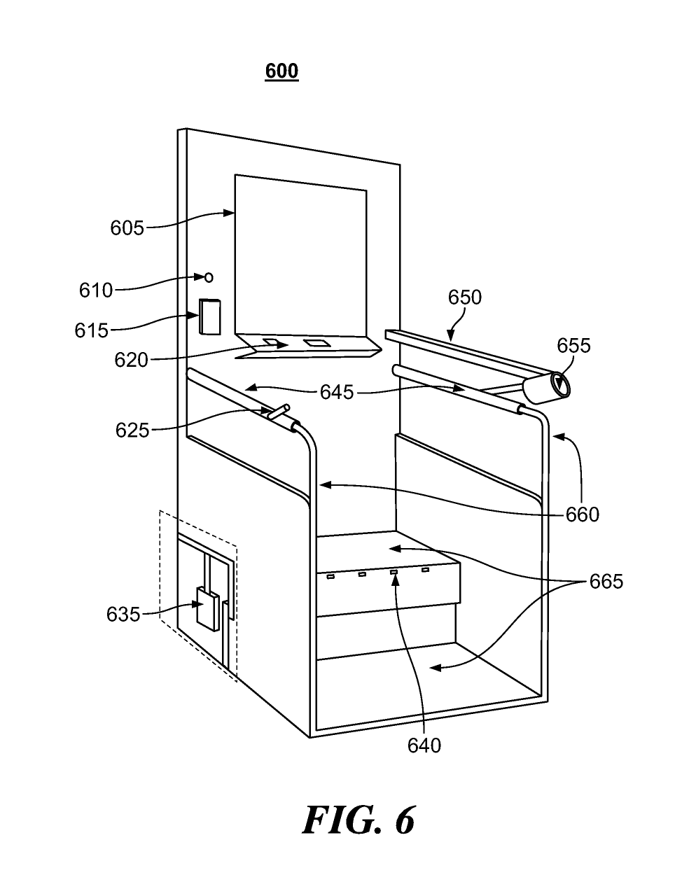

In an embodiment as shown in FIG. 6, the wellness testing device 10 may be described with respect to fitometer 600 (a measurement device for testing fitness level that returns a numeric fitness score using algorithms incorporating, in one embodiment, the number of steps performed at an individualized step height and the pulse rate after completing the steps at time zero, one and two minutes). The fitometer 600 may include a visual output unit 605. The visual output unit 605 may be, for example, a flat-screen display such as a LCD (Liquid Crystal Display) or LED (Light-Emitting Diode) display device or any device capable of conveying visual information to a user of the fitometer 600. In one embodiment, the fitometer 600 may include an image capture device 610. The image capture device 610 may be used to capture images or video during operation of the fitometer 600. Images or video captured during operation of the fitometer 600 may be stored in a storage memory of the fitometer 600, or may be remotely stored. Additionally, images or video captured by the image capture device 610 may be viewed remotely from the fitometer 600 by, for example, transmitting the captured images or video across a private network or a public network such as the Internet.

During operation of the fitometer 600, an audio output device 615 may be used to convey audible information to a user. For example, the audio output device 615 may present audible instructions, testing information, and/or encouragement to the user. Additionally, the audio output device 615 may convey audible information from a remotely located test administrator to the user. An interaction panel 620 may be used by a user or test administrator to enter information used or stored by the fitometer 600. The fitometer 600 may use a biological data input device 625 to obtain biological data from a user during operation. In one embodiment, the biological data input device 625 may include, for example, a finger pulse oximeter connected to a user's finger for measuring a user's heart rate.

The fitometer 600 may include an overlapping step mechanism 635 for varying step height during operation. Additionally, the fitometer may include a video capture mechanism 640 for capturing the toe height and acceleration of a user's toe. The video capture mechanism 640 may be used as a safety mechanism for the user. In order to detect a user's placement on and speed of use of the fitometer 600, pressure sensors 665 may be used. The pressure sensors 665 may be used, for example, to measure the weight of a user, to measure the speed at which the user is performing a wellness test, and/or to verify that the users is properly performing the fitness test.

To provide stability for users and to measure the user's pulse, the fitometer 600 may include hand rails 660. In one embodiment, the hand rails 660 may be weight-sensitive and may be used to detect a total weight load on the hand rails 660 (in order to ensure that users do not make the test easier by leaning on the rails). The hand rails 600 may further include hand electrode pulsometers 645 for measuring the user's pulse. In one embodiment, the fitometer 600 may include a stabilizing bar 650 attached to a wrist pulsometer 655. The wrist pulsometer 655 may be, for example, a piezoelectric wrist sensor.

It should be understood that various changes and modifications to the presently preferred embodiments described herein will be apparent to those skilled in the art. Such changes and modifications can be made without departing from the spirit and scope of the present disclosure and without diminishing its intended advantages. It is therefore intended that such changes and modifications be covered by the appended claims.

We claim:

1. A method comprising:
performing a baseline assessment on a user of all measurable, modifiable and meaningful wellness factors;
generating a baseline wellness score that is proportional to each wellness factor's contribution to modifiable risk for adverse events from the analyzed wellness factors using a computer that implements an algorithm;
providing an incentive to the user based on the user's baseline wellness score;
performing a subsequent assessment of one or all of the wellness factors on the user at a later date;
generating a subsequent wellness score from the analyzed subsequent performance test using the computer;
comparing the contribution of each wellness factor to the baseline wellness score and to the subsequent wellness score; and
providing an additional incentive (that is proportional to the impact of the wellness factor change on the risk for adverse events multiplied by the degree of difficulty, on average, in making the wellness factor change) to the user if incentive conditions are improved as shown by the comparison between the contribution of each wellness factor to the baseline wellness score and the contribution of each to the subsequent wellness score.

2. The method of claim 1, wherein the wellness factors comprise body mass index (weight divided by the square of the height), blood pressure, fitness level, cholesterol level, and whether the user consumes nicotine products.

3. The method of claim 2, wherein the fitness level is tested using a three minute step test of variable step height, variable step completion, and the capturing of biometric data of pulse rate at various times after the step test is completed and using an algorithm on a computer to determine a numeric step test score.

4. The method of claim 1, wherein the incentive is a monetary incentive of sufficient frequency (in one embodiment monthly), connectedness in time (in one embodiment <6 weeks) and magnitude (in one embodiment >2% of income for >50% of employees) to induce sustained behavior change.

5. The method of claim 1, wherein the incentive is delivered with individualized messaging determined by the current wellness factors, the recent and remote changes in wellness factors and the goal(s) the user selects.

6. The method of claim 1, wherein messaging and other incentive conditions correspond with wellness goals.

7. An apparatus comprising:
a stepper device; and
a processor connected to the stepper device, configured to:
measure biometric data of a user;
analyze fitness based on the measured fitness related data; and
generate a user fitness score using the analyzed measured fitness related data.

8. The apparatus of claim 7, wherein the stepper device comprises height-adjusting mats for tailoring an exercise environment based on the user's physical characteristics or capabilities.

9. The apparatus of claim 7, wherein the apparatus is capable of transmitting user fitness score and other wellness data to a server.

10. The apparatus of claim 7, wherein the server executes a wellness score engine to calculate a user's wellness score and stores results in a member wellness score database.

11. The apparatus of claim 7, wherein a wellness test administrator enters user liveliness data into a computer and transmits the measured wellness data to the server.

12. The apparatus of claim 7, wherein the wellness factors comprise Body Mass Index (weight over height squared), blood pressure, fitness level, cholesterol level, and whether the user consumes nicotine.

13. A method comprising:
a computer for supporting an internet-based portal which performs operations of;
receiving wellness data associated with a user,
presenting the user with information about the user's wellness including the proportional contribution of each wellness factor on their modifiable risk for adverse events;
tracking the amount of cash available to incentivize users to make changes
presenting the user with options for attaining the available cash, with cash incentives proportional to risk reduction multiplied by the difficulty of change
tracking incentives related to the options chosen presented as user goals;
displaying the user goals and progress towards the user goals;
displaying a personalized evaluation of the user's goals and encouragement for the user to continue towards the user's goal; and
presenting the user with additional user goals that may be selected by the user.

14. The method of claim 13, wherein the wellness data associated with a user is input by a third party wellness test administrator.

15. The method of claim 13, wherein the displaying user goals and progress towards the user goals is performed by using a user-modifiable graphic that provides the user with information about the selected goal and incentive information, and changes the selected goal and incentive information based on user input.

16. The method of claim 13, wherein the internet-based portal provides a take action button for quickly selecting a suggested new goal resulting in changes in subsequent messaging and reminders.
